(19) United States
(12) Patent Application Publication
(10) Pub. No.: US 2024/0415760 A1
(43) Pub. Date: Dec. 19, 2024

(54) PHOTOCURABLE COMPOSITION

(71) Applicant: THREEBOND CO., LTD., Hachioji-shi, Tokyo (JP)

(72) Inventors: Yumi MORIKAWA, Hachioji-shi, Tokyo (JP); Natsumi HARADA, Hachioji-shi, Tokyo (JP)

(21) Appl. No.: 18/704,300

(22) PCT Filed: Oct. 31, 2022
(86) PCT No.: PCT/JP2022/040729

§ 371 Date: Apr. 24, 2024

(30) Foreign Application Priority Data
Nov. 12, 2021 (JP) 2021-184527

(51) Int. Cl. A61K 8/87; A61K 8/46; A61K 8/55; A61Q 3/02; C08G 18/12; C08G 18/38; C08G 18/67; C08K 3/32; C08K 5/092; C08K 5/5333

(52) U.S. Cl. CPC A61K 8/87; A61K 8/46; A61K 8/55; A61Q 3/02; C08G 18/12; C08G 18/3876; C08G 18/672; C08K 3/32; C08K 5/092; C08K 5/5333; A61K 2800/81; C08K 2003/329

(57) ABSTRACT

It has been conventionally difficult to achieve both storage stability and photocurability by addition of a polythiol compound to a compound having a (meth)acryloyl group. A photocurable composition contains components (A) to (D): Component (A): a compound having a (meth)acryloyl group, Component (B): a polythiol compound, Component (C): a photoinitiator, Component (D): a storage stabilizer having pKa of 1.0 to 4.0 (containing no component (A)).

### TECHNICAL FIELD

The present invention relates to a photocurable composition comprising a thiol compound.

### BACKGROUND ART

It has been known that surface curability is enhanced by addition of a polythiol compound to a compound having a (meth)acryloyl group. The reactivity is basically high and thus storage stability tends to be reduced. A polymerization inhibitor used in International Publication No. WO 2017/154428 (corresponding to US Patent Publication No. 2019/0040175) can be added to suppress radical polymerization and increase storage stability.

### SUMMARY OF INVENTION

However, there is a disadvantage that photocurability is also reduced, and storage stability and curability are in a trade-off relationship. It has been conventionally difficult to achieve both storage stability and photocurability in the case of adding a polythiol compound to a compound having a (meth)acryloyl group.

Therefore, the present invention has been made in view of the above circumstances, and an object thereof is to provide a photocurable composition having both storage stability and photocurability.

The present inventors have made intensive studies in order to achieve the above object, and as a result, have completed the present invention, which is a photocurable composition.

The gist of the present invention will be described below. A first aspect of the present invention relates to a photocurable composition comprising component (A) to component (D):
Component (A): a compound having a (meth)acryloyl group
Component (B): a polythiol compound
Component (C): a photoinitiator
Component (D): a storage stabilizer having pKa of 1.0 to 4.0 (excluding the component (A)).

A second aspect of the present invention relates to the photocurable composition according to the first aspect, wherein the component (D) is a phosphorus compound.

A third aspect of the present invention relates to the photocurable composition according to the second aspect, wherein the phosphorus compound is a phosphonic acid compound, R¹—P(═O)(OR²)₂, wherein R¹ is a hydrogen atom, or an organic group not bound to a phosphorus atom via an oxygen atom, and each R² is independently a hydrogen atom or an organic group.

A fourth aspect of the present invention relates to the photocurable composition according to the first aspect, wherein the component (D) is at least one selected from the group consisting of phosphoric acid, oxalic acid, 2-ethylhexyl (2-ethylhexyl)phosphonate, phenylphosphonic acid, vinylphosphonic acid and methylphosphonic acid.

A fifth aspect of the present invention relates to the photocurable composition according to any of the first aspect to the fourth aspect, the composition comprising 0.01 to 10.0% by mass of the component (D) relative to the entire composition.

A sixth aspect of the present invention relates to the photocurable composition according to any of the first aspect to the fifth aspect, wherein the composition does not comprise any storage stabilizer other than the component (D).

A seventh aspect of the present invention relates to the photocurable composition according to any of the first aspect to the sixth aspect, the composition comprising 0.1 to 50 parts by mass of the component (B) and 0.1 to 10 parts by mass of the component (C), based on 100 parts by mass of the component (A).

An eighth aspect of the present invention relates to the photocurable composition according to any of the first aspect to the seventh aspect, wherein the component (A) comprises a (meth)acrylate oligomer and a (meth)acrylate monomer.

A ninth aspect of the present invention relates to the photocurable composition according to the eighth aspect, wherein the (meth)acrylate monomer consists only of a monofunctional (meth)acrylate and/or a difunctional (meth)acrylate.

A tenth aspect of the present invention relates to the photocurable composition according to the ninth aspect, wherein the monofunctional (meth)acrylate is a monofunctional (meth)acrylate having a hydroxyl group.

An eleventh aspect of the present invention relates to the photocurable composition according to the ninth aspect or the tenth aspect, wherein the difunctional (meth)acrylate is dimethylol tricyclodecane diacrylate.

A twelfth aspect of the present invention relates to the photocurable composition according to any of the first aspect to the eleventh aspect, for use in a nail or an artificial nail.

A thirteenth aspect of the present invention relates to the photocurable composition for use in a nail or an artificial nail according to the twelfth aspect, wherein the photocurable composition is for a topcoat (the photocurable composition according to any of the first aspect to the eleventh aspect is a photocurable composition for a topcoat for use in a nail or an artificial nail).

### DESCRIPTION OF EMBODIMENTS

A first aspect of the present invention relates to a photocurable composition comprising the following component (A) to component (D):
Component (A): a compound having a (meth)acryloyl group;
Component (B): a polythiol compound;
Component (C): a photoinitiator; and
Component (D): a storage stabilizer having pKa in the range of 1.0 to 4.0 (excluding the component (A)).

The above configuration can provide a photocurable composition comprising a compound having a (meth)acryloyl group and a polythiol compound, in which not only an increase in storage stability, but also no reduction in curability is achieved. In other words, the above configuration can provide a photocurable composition capable of having both storage stability and photocurability.

As used herein, the photocurable composition is also simply referred to as “composition” or “the composition according to the present invention”.

Hereinafter, preferred embodiments of the present invention will be described. The present invention is not limited only to the following embodiments, and can be variously modified within the scope of claims. Any embodiment described herein can be optionally combined and thus serve as any other embodiment.

Throughout the description, unless particularly stated otherwise, any expression in a singular form should be understood to encompass the concept of its plural form. Therefore, unless particularly stated otherwise, the article specifying a single form (for example, “a”, “an”, “the”, and the like in the case of English language) should be understood to encompass the concept of its plural form. Further, unless particularly stated otherwise, any term used in the present description should be understood as a term that is used to have the meaning conventionally used in the relevant technical field. Therefore, unless defined otherwise, all the technical terms and scientific terms used in the present description have the same meaning as generally understood by a person ordinarily skilled in the art to which the present invention is pertained. If there is any conflict in meaning, the present description (including the definitions) takes priority.

As used herein, the term “(meth)acrylic” encompasses both acrylic and methacrylic. Therefore, for example, the term “(meth)acrylic acid” encompasses both acrylic acid and methacrylic acid. Similarly, the term “(meth)acryloyl” encompasses both acryloyl and methacryloyl. Therefore, for example, the term “(meth)acryloyl group” encompasses both “acryloyl group” and “methacryloyl group”.

As used herein, “A and/or B” means both A and B, or any one of A or B.

The present invention will be described in detail below. The component (A) usable in the present invention can be any compound having a (meth)acryloyl group. Specifically, the component (A) refers to a compound such as (meth)acrylate or (meth)acrylamide, and also encompasses a (meth)acrylate monomer and a (meth)acrylate oligomer. The component (A) preferably comprises a (meth)acrylate oligomer and a (meth)acrylate monomer, more preferably consists only of a (meth)acrylate oligomer and a (meth)acrylate monomer. In the embodiment, the (meth)acrylate monomer preferably comprises a monofunctional (meth)acrylate and/or a difunctional (meth)acrylate, more preferably consists only of a monofunctional (meth)acrylate and/or a difunctional (meth)acrylate, particularly preferably consists only of a monofunctional (meth)acrylate and a difunctional (meth)acrylate. Hereinafter, “acryloyl” and “methacryloyl” are collectively called as (meth)acryloyl, and “compound having a (meth)acryloyl group” is also called as “(meth)acrylate”. The component (A) is preferably in the form of a liquid under an atmosphere at 25° C., and can be used as long as it has favorable compatibility with the following component (B) and component (C) in the present invention.

Specific examples of the (meth)acrylate oligomer may include a (meth)acrylate oligomer having an ester bond in its molecule, a (meth)acrylate oligomer having an ether bond in its molecule, a (meth)acrylate oligomer having a urethane bond in its molecule, and an epoxy-modified (meth)acrylate oligomer, and examples of the main backbone thereof may include bisphenol A, novolac phenol, polybutadiene, polyester, and polyether, but not limited thereto. The component (A) usable in the present invention also encompasses a compound having one or more epoxy groups and one or more (meth)acryloyl groups in one molecule. The component (A) preferably contains a (meth)acrylate oligomer containing three or more (meth)acryloyl groups in one molecule (having the number of functional groups of 3 or more) in order to enhance surface curability. In a case where the component (A) contains a (meth)acrylate oligomer having three or more (meth)acryloyl groups in one molecule (having the number of functional groups of 3 or more), the upper limit of the number of (meth)acryloyl groups (the number of functional groups) contained in one molecule of the (meth)acrylate oligomer is preferably 10 or less.

As a method for synthesizing the (meth)acrylate oligomer having an ester bond, a synthesis method which includes forming an ester bond with a polyol and a polyvalent carboxylic acid and adding acrylic acid to an unreacted hydroxyl group has been known, but not limited to this synthesis method. Specific examples thereof may include Aronix M-6100, M-6200, M-6250, M-6500, M-7100, M-7300K, M-8030, M-8060, M-8100, M-8530, M-8560, and M-9050 manufactured by Toagosei Co., Ltd., and UV-3500BA, UV-3520TL, UV-3200B, and UV-3000B manufactured by Nippon Synthetic Chemical Industry Co., Ltd., but not limited thereto.

As a method for synthesizing the (meth)acrylate oligomer having an ether bond, a synthesis method which includes adding acrylic acid to a hydroxyl group of a polyether polyol, or a hydroxyl group of an aromatic polyether polyol such as bisphenol has been known, but not limited to this synthesis method. Specific examples thereof may include UV-6640B, UV-6100B, UV-3700B, and the like manufactured by Nippon Synthetic Chemical Industry Co., Ltd., Light (meth)acrylates 3EG-A, 4EG-A, 9EG-A, 14EG-A, PTMGA-250, BP-4EA, BP-4PA, BP-10EA, and the like manufactured by Kyoeisha Chemical Co., Ltd., EBECRYL3700 and the like manufactured by Daicel-Cytec Co., Ltd., and KY-11 manufactured by Negami Chemical Industrial Co., Ltd., but not limited thereto.

As a method for synthesizing the (meth)acrylate oligomer having a urethane bond, for example, a synthesis method which includes forming a urethane bond by a polyol and a polyisocyanate and adding a compound having a hydroxyl group and a (meth)acryloyl group in its molecule, or (meth)acryloyl acid to the remaining isocyanate group has been known. The (meth)acrylate oligomer having a urethane bond is preferably added from the viewpoint of an enhancement in durability. Specific examples may include AH-600, AT-600, UA-306H, and UF-8001G manufactured by Kyoeisha Chemical Co., Ltd., UN-3320HA, and UN-904 manufactured by Negami Chemical Industrial Co., Ltd., but not limited thereto.

The epoxy-modified (meth)acrylate oligomer can be synthesized by subjecting a glycidyl group of a polyfunctional glycidyl ether compound to ring-opening polymerization with (meth)acrylic acid or the like, without limitation to such synthesis. Various types of backbones such as a bisphenol A type, a bisphenol F type, and a novolac phenol type can be used in a main chain of such a polyfunctional glycidyl ether. Specific examples of the epoxy-modified acrylic oligomer may include epoxy esters 3000A and 3002A manufactured by Kyoeisha Chemical Co., Ltd., and EBECRYL3700 manufactured by Daicel-Allnex Ltd., but not limited thereto.

A weight average molecular weight (or molecular weight) of the (meth)acrylate oligomer is preferably 1000 to 50000. A weight average molecular weight of 1000 or more can allow a cured product to exhibit toughness, and a weight average molecular weight of 50000 or less can allow viscosity of the composition to be kept low. The weight average molecular weight (or molecular weight) of the (meth)acrylate oligomer is more preferably more than 1000 and 10000 or less, particularly preferably 1500 to 5000. The weight average molecular weight as used herein refers to a weight average molecular weight in terms of polystyrene, measured by gel permeation chromatography.

The (meth)acrylate monomer can include monofunctional, difunctional, or trifunctional (meth)acrylate monomer and (meth)acrylamide monomer. A plurality of other monomers can also be used in combination.

Specific examples of the monofunctional (meth)acrylate monomer may include lauryl (meth)acrylate, stearyl (meth)acrylate, ethyl carbitol (meth)acrylate, tetrahydrofurfuryl (meth)acrylate, caprolactone-modified tetrahydrofurfuryl (meth)acrylate, cyclohexyl (meth)acrylate, dicyclopentanyl (meth)acrylate, isobornyl (meth)acrylate, benzyl (meth)acrylate, phenyl (meth)acrylate, phenoxyethyl (meth)acrylate, phenoxydiethylene glycol (meth)acrylate, phenoxytetraethylene glycol (meth)acrylate, nonylphenoxyethyl (meth)acrylate, nonylphenoxytetraethylene glycol (meth)acrylate, methoxydiethylene glycol (meth)acrylate, ethoxydiethylene glycol (meth)acrylate, butoxyethyl (meth)acrylate, butoxytriethylene glycol (meth)acrylate, 2-ethylhexylpolyethylene glycol (meth)acrylate, 4-hydroxybutyl (meth)acrylate, nonylphenylpolypropylene glycol (meth)acrylate, methoxydipropylene glycol (meth)acrylate, 2-hydroxyethyl (meth)acrylate, 2-hydroxypropyl (meth)acrylate, glycerol (meth)acrylate, polyethylene glycol (meth)acrylate, polypropylene glycol (meth)acrylate, epichlorohydrin-modified butyl (meth)acrylate, epichlorohydrin-modified phenoxy (meth)acrylate, N,N-dimethylaminoethyl (meth)acrylate, and N,N-diethylaminoethyl (meth)acrylate, but not limited thereto. The component (A) preferably contains a monofunctional monomer having a hydroxyl group. In other words, in a preferred embodiment of the present invention, the monofunctional (meth)acrylate is monofunctional (meth)acrylate having a hydroxyl group. Specific examples may include 4-hydroxybutyl (meth)acrylate, 2-hydroxyethyl (meth)acrylate, and 2-hydroxypropyl (meth)acrylate, but not limited thereto. The monofunctional (meth)acrylate is preferably at least one selected from the group consisting of 2-hydroxyethyl (meth)acrylate and 2-hydroxypropyl (meth)acrylate, more preferably 2-hydroxypropylacrylate and/or 2-hydroxypropyl methacrylate.

Examples of the monofunctional (meth)acrylate monomer also may include a (meth)acrylate monomer having an acidic group. The monofunctional (meth)acrylate monomer particularly refers to carboxylic acid or phosphoric acid having a (meth)acryloyl group in its molecule. Examples of the carboxylic acid having a (meth)acryloyl group in its molecule may include (meth)acryloyl acid, 3-(meth)acryloyloxypropylsuccinic acid, 4-(meth)acryloyloxybutylsuccinic acid, 2-(meth)acryloyloxyethylmaleic acid, 3-(meth)acryloyloxypropylmaleic acid, 4-(meth)acryloyloxybutylmaleic acid, 2-(meth)acryloyloxyethylhexahydrophthalic acid, 3-(meth)acryloyloxypropylhexahydrophthalic acid, 4-(meth)acryloyloxybutylhexahydrophthalic acid, 2-(meth)acryloyloxyethylphthalic acid, 3-(meth)acryloyloxypropylphthalic acid, and 4-(meth)acryloyloxybutylphthalic acid, but not limited thereto. Examples of the phosphoric acid having a (meth)acryloyl group in its molecule may include 2-ethylhexyl acid phosphate, 2-hydroxyethyl methacrylate acid phosphate, and dibutyl phosphate, but not limited thereto. The (meth)acrylate monomer having an acidic group is preferably contained for the purpose of an enhancement in durability.

Specific examples of the difunctional (meth)acrylate monomer may include 1,3-butylene glycol di(meth)acrylate, 1,4-butylene glycol di(meth)acrylate, tricyclodecane dimethanol di(meth)acrylate, neopentyl glycol di(meth)acrylate, 1,6-hexane glycol di(meth)acrylate, ethylene glycol diacrylate, tetraethylene glycol di(meth)acrylate, polyethylene glycol di(meth)acrylate, propylene glycol di(meth)acrylate, tripropylene glycol di(meth)acrylate, dimethylol tricyclodecane di(meth)acrylate, ethylene oxide-modified neopentyl glycol di(meth)acrylate, propylene oxide-modified neopentyl glycol di(meth)acrylate, bisphenol A di(meth)acrylate, ethylene oxide-modified bisphenol A di(meth)acrylate, epichlorohydrin-modified bisphenol A di(meth)acrylate, ethylene oxide-modified bisphenol S di(meth)acrylate, neopentyl glycol-modified trimethylolpropane di(meth)acrylate, dicyclopentenyl di(meth)acrylate, ethylene oxide-modified dicyclopentenyl di(meth)acrylate, and diacryloyl isocyanurate, but not limited thereto. In consideration of an enhancement in curability, dimethylol tricyclodecane di(meth)acrylate is preferably used, and dimethylol tricyclodecane diacrylate is more preferably used. In other words, in a preferred embodiment of the present invention, the difunctional (meth)acrylate monomer is dimethylol tricyclodecane di(meth)acrylate. In a more preferred embodiment of the present invention, the difunctional (meth)acrylate monomer is dimethylol tricyclodecane diacrylate.

Specific examples of the trifunctional (meth)acrylate monomer may include trimethylolpropane tri(meth)acrylate, trimethylolpropane tri(meth)acrylate, pentaerythritol tri(meth)acrylate, EO-modified trimethylolpropane tri(meth)acrylate, PO-modified trimethylolpropane tri(meth)acrylate, ECH-modified trimethylolpropane tri(meth)acrylate, ECH-modified glycerol tri(meth)acrylate, and tris(acryloyloxyethyl)isocyanurate, but not limited thereto.

Specific examples of the (meth)acrylamide monomer may include dimethyl(meth)acrylamide, (meth)acryloylmorpholine, and diethyl(meth)acrylamide, but not limited thereto. Although a reason cannot be clearly found, the monomer preferably contains the (meth)acrylamide monomer from the viewpoint of an enhancement in durability. Specific examples of the (meth)acrylamide monomer in the present invention may include DMAA, ACMO, DEAA, and the like manufactured by KJ Chemicals Corporation, but not limited thereto.

The component (A) preferably contains both the (meth)acrylate oligomer and the (meth)acrylate monomer. In a case where the component (A) comprises the (meth)acrylate oligomer and the (meth)acrylate monomer, a ratio of the oligomer and the monomer (mass ratio of (meth)acrylate oligomer:(meth)acrylate monomer) is preferably 50:50 to 95:5, more preferably 55:45 to 65:35. The (meth)acrylate oligomer is contained to allow an enhancement in durability to be exhibited. In a case where the (meth)acrylate oligomer is a mixture of two or more kinds thereof, the ratio is referred to as a ratio of the total amount of such (meth)acrylate oligomers. Similarly, in a case where the (meth)acrylate monomer is a mixture of two or more kinds thereof, the ratio is referred to as a ratio of the total amount of such (meth)acrylate monomers.

The (meth)acrylate monomer is preferably a mixture of monofunctional one and di- or higher difunctional one, more preferably a mixture of monofunctional one and difunctional one (namely, it comprises a monofunctional (meth)acrylate and a difunctional (meth)acrylate), particularly preferably consists only of a monofunctional (meth)acrylate and a difunctional (meth)acrylate. A mixing ratio of the monofunctional (meth)acrylate and the difunctional (meth)acrylate (mass ratio of monofunctional (meth)acrylate:difunctional (meth)acrylate) is preferably 50:50 to 90:10, more preferably 70:30 to 80:20. In a case where the monofunctional (meth)acrylate is a mixture of two or more kinds thereof, the ratio is referred to as a ratio of the total amount of such monofunctional (meth)acrylates. Similarly, in a case where the difunctional (meth)acrylate is a mixture of two or more kinds thereof, the ratio is referred to as a ratio of the total amount of such difunctional (meth)acrylates. The mixing ratio is particularly preferably adopted in a case where the component (A) consists only of the (meth)acrylate oligomer and the (meth)acrylate monomer, and the (meth)acrylate monomer consists only of the monofunctional (meth)acrylate and the difunctional (meth)acrylate.

The component (B) usable in the present invention is a polythiol compound. The component (B) is not particularly limited as long as it has two or more thiol groups, and may be used singly or in combinations of two or more kinds thereof. Specific examples of the component (B) may include an aliphatic polythiol compound, an aromatic polythiol compound, and a polythiol compound having a sulfide bond, but not limited thereto.

Examples of an aliphatic polythiol compound having two thiol groups may include 1,2-ethanedithiol, 1,2-propanedithiol, 1,3-propanedithiol, 1,4-butanedithiol, 1,6-hexanedithiol, 1,7-heptanedithiol, 1,8-octanedithiol, 1,9-nonanedithiol, 1,10-decanedithiol, 1,12-dodecanedithiol, 2,2-dimethyl-1,3-propanedithiol, 3-methyl-1,5-pentanedithiol, 2-methyl-1,8-octanedithiol, 1,4-cyclohexanedithiol, 1,4-bis(mercaptomethyl)cyclohexane, 1,1-cyclohexanedithiol, 1,2-cyclohexanedithiol, bicyclo[2,2,1]hepta-exo-cis-2,3-dithiol, 1,1-bis(mercaptomethyl)cyclohexane, bis(2-mercaptoethyl)ether, ethylene glycol bis(2-mercaptoacetate), and ethylene glycol bis(3-mercaptopropionate), but not limited thereto.

Examples of an aliphatic polythiol compound having three thiol groups may include 1,1,1-tris(mercaptomethyl) ethane, 2-ethyl-2-mercaptomethyl-1,3-propanedithiol, 1,2,3-propanetrithiol, trimethylolpropane tris(2-mercaptoacetate), trimethylolpropane tris(3-mercaptopropionate), and tris[(mercaptopropynyloxy)-ethyl]isocyanurate, but not limited thereto.

Examples of an aliphatic polythiol compound having four or more thiol groups may include pentaerythritol tetrakis(2-mercaptoacetate), pentaerythritol tetrakis(3-mercaptopropionate), pentaerythritol tetrakis(3-mercaptobutyrate), and dipentaerythritol hexakis (3-mercaptopropionate), but not limited thereto.

Examples of the aromatic polythiol compound may include 1,2-dimercaptobenzene, 1,3-dimercaptobenzene, 1,4-dimercaptobenzene, 1,2-bis(mercaptomethyl)benzene, 1,3-bis(mercaptomethyl)benzene, 1,4-bis(mercaptomethyl)benzene, 1,2-bis(2-mercaptoethyl)benzene, 1,3-bis(2-mercaptoethyl)benzene, 1,4-bis(2-mercaptoethyl)benzene, 1,2-bis(2-mercaptoethyleneoxy)benzene, 1,3-bis(2-mercaptoethyleneoxy)benzene, 1,4-bis(2-mercaptoethyleneoxy)benzene, 1,2,3-trimercaptobenzene, 1,2,4-trimercaptobenzene, 1,3,5-trimercaptobenzene, 1,2,3-tris(mercaptomethyl)benzene, 1,2,4-tris(mercaptomethyl)benzene, 1,3,5-tris(mercaptomethyl)benzene, 1,2,3-tris(2-mercaptoethyl)benzene, 1,2,4-tris(2-mercaptoethyl)benzene, 1,3,5-tris(2-mercaptoethyl)benzene, 1,2,3-tris(2-mercaptoethyleneoxy)benzene, 1,2,4-tris(2-mercaptoethyleneoxy)benzene, 1,3,5-tris(2-mercaptoethyleneoxy)benzene, 1,2,3,4-tetramercaptobenzene, 1,2,3,5-tetramercaptobenzene, 1,2,4,5-tetramercaptobenzene, 1,2,3,4-tetrakis(mercaptomethyl)benzene, 1,2,3,5-tetrakis(mercaptomethyl)benzene, 1,2,4,5-tetrakis(mercaptomethyl)benzene, 1,2,3,4-tetrakis(2-mercaptoethyl)benzene, 1,2,3,5-tetrakis(2-mercaptoethyl)benzene, 1,2,4,5-tetrakis(2-mercaptoethyl)benzene, 1,2,3,4-tetrakis(2-mercaptoethyleneoxy)benzene, 1,2,3,5-tetrakis(2-mercaptoethyleneoxy)benzene, 1,2,4,5-tetrakis(2-mercaptoethyleneoxy)benzene, 2,2′-mercaptobiphenyl, 4,4′-thiobis-benzenethiol, 4,4′-dimercaptobiphenyl, 4,4′-dimercaptobibenzyl, 2,5-toluenedithiol, 3,4-toluenedithiol, 1,4-naphthalenedithiol, 1,5-naphthalenedithiol, 2,6-naphthalenedithiol, 2,7-naphthalenedithiol, 2,4-dimethylbenzene-1,3-dithiol, 4,5-dimethylbenzene-1,3-dithiol, 9,10-anthracenedimethanethiol, 1,3-bis(2-mercaptoethylthio)benzene, 1,4-bis(2-mercaptoethylthio)benzene, 1,2-bis(2-mercaptoethylthiomethyl)benzene, 1,3-bis(2-mercaptoethylthiomethyl)benzene, 1,4-bis(2-mercaptoethylthiomethyl)benzene, 1,2,3-tris(2-mercaptoethylthio)benzene, 1,2,4-tris(2-mercaptoethylthio)benzene, 1,3,5-tris(2-mercaptoethylthio)benzene, 1,2,3,4-tetrakis(2-mercaptoethylthio)benzene, 1,2,3,5-tetrakis(2-mercaptoethylthio)benzene, and 1,2,4,5-tetrakis(2-mercaptoethylthio)benzene, but not limited thereto.

Examples of the polythiol compound having a sulfide bond may include bis(2-mercaptoethyl)sulfide, bis(2-mercaptoethylthio)methane, 1,2-bis(2-mercaptoethylthio)ethane, 1,3-bis(2-mercaptoethylthio)propane, 1,2,3-tris(2-mercaptoethylthio)propane, tetrakis(2-mercaptoethylthiomethyl)methane, 1,2-bis(2-mercaptoethylthio)propanethiol, 2,5-dimercapto-1,4-dithiane, bis(2-mercaptoethyl)disulfide, 3,4-thiophenedithiol, 1,2-bis(2-mercaptoethyl)thio-3-mercaptopropane, and bis-(2-mercaptoethylthio-3-mercaptopropane)sulfide, but not limited thereto.

Specific examples of the component (B) having a secondary thiol group may include pentaerythritol tetrakis(3-mercaptobutyrate), 1,4-bis(3-mercaptobutyryloxy)butane, 1,3,5-tris(3-mercaptobutyryloxyethyl)-1,3,5-triazin-2,4,6 (1H,3H,5H)-trione, trimethylolpropane tris(3-mercaptobutyrate), trimethylolethane tris(3-mercaptobutyrate), trimethylolpropane tris(3-mercaptobutyrate), and trimethylolethane tris(3-mercaptobutyrate), but not limited thereto. Examples of a commercial product may include PEMP manufactured by SC Organic Chemical Co., Ltd., and KarenzMT (registered trademark) series, PE1, BD1, and NR1 manufactured by Showa Denko K.K., but not limited thereto.

A content of the component (B) is preferably 0.1 to 50 parts by mass based on 100 parts by mass of the component (A). The content of the component (B) is particularly preferably 1 to 30 parts by mass, most preferably 10 to 20 parts by mass, based on 100 parts by mass of the component (A). The content of the component (B) is 0.1 parts by mass or more to result in an enhancement in surface curability, and is 50 parts by mass or less to result in an enhancement in storage stability. In a case where the component (B) is a mixture of two or more kinds thereof, the ratio is referred to as a ratio of the total content of such components (B).

The component (C) usable in the present invention is a photoinitiator. The component (C) is not limited as long as it is a radical photoinitiator which generates radical species by an energy ray such as visible light, ultraviolet light, X-ray, or electron beam.

Specific examples of the component (C) may include acetophenones such as diethoxyacetophenone, 2-hydroxy-2-methyl-1-phenylpropan-1-one, benzyl dimethyl ketal, 4-(2-hydroxyethoxy)phenyl-(2-hydroxy-2-propyl)ketone, 1-hydroxycyclohexyl phenyl ketone, 2-methyl-2-morpholino(4-thiomethylphenyl)propan-1-one, 2-benzyl-2-dimethylamino-1-(4-morpholino phenyl)butanone, and a 2-hydroxy-2-methyl-1-[4-(1-methylvinyl)phenyl]propanone oligomer; benzoins such as benzoin, benzoin methyl ether, benzoin ethyl ether, benzoin isopropyl ether, and benzoin isobutyl ether; benzophenones such as benzophenone, methyl o-benzoylbenzoate, 4-phenylbenzophenone, 4-benzoyl-4′-methyl-diphenyl sulfide, 3,3′,4,4′-tetra(t-butylperoxycarbonyl)benzophenone, 2,4,6-trimethylbenzophenone, 4-benzoyl-N,N-dimethyl-N-[2-(1-oxo-2-propenyloxy)ethyl]benzenemethanaminium bromide, and (4-benzoylbenzyl)trimethylammonium chloride; and thioxanthones such as 2-isopropylthioxanthone, 4-isopropylthioxanthone, 2,4-diethylthioxanthone, 2,4-dichlorothioxanthone, 1-chloro-4-propoxythioxanthone, and 2-(3-dimethylamino-2-hydroxy)-3,4-dimethyl-9H-thioxanthon-9-one-mesochloride, but not limited thereto. A plurality of the components (C) can be used in combinations thereof.

The component (C) is added in an amount of 0.1 to 20 parts by mass, further preferably 0.1 to 10 parts by mass, particularly preferably 2 to 4 parts by mass, based on 100 parts by mass of the component (A). In a case where more than 0.1 parts by mass of the component (C) is added, photocurability can be maintained. On the other hand, in a case where less than 20 parts by mass of the component (C) is added, storage stability can be maintained without thickening during storage. In a case where the component (C) is a mixture of two or more kinds thereof, it is referred to as a ratio of the total amount of such components (C).

In a preferred embodiment of the present invention, the component (B) is contained in an amount of 0.1 to 50 parts by mass and the component (C) is contained in an amount of 0.1 to 20 parts by mass, based on 100 parts by mass of the component (A). In a more preferred embodiment of the present invention, the component (B) is contained in an amount of 0.1 to 50 parts by mass and the component (C) is contained in an amount of 0.1 to 10 parts by mass, based on 100 parts by mass of the component (A). In a further preferred embodiment of the present invention, the component (B) is contained in an amount of 1 to 30 parts by mass and the component (C) is contained in an amount of 0.1 to 10 parts by mass, based on 100 parts by mass of the component (A). In a particularly preferred embodiment of the present invention, the component (B) is contained in an amount of 10 to 20 parts by mass and the component (C) is contained in an amount of 2 to 4 parts by mass, based on 100 parts by mass of the component (A).

A visible light type photoinitiator, instead of or in addition to the photoinitiator, is preferably contained in the component (C), the component (C) more preferably contains the photoinitiator and a visible light type photoinitiator, and the component (C) further preferably consists only of the photoinitiator and a visible light type photoinitiator. In the embodiment, a content of the visible light type photoinitiator relative to the entire component (C) is preferably 0 to 70% by mass, more preferably 40 to 60% by mass, particularly preferably 45 to 55% by mass. Alternatively, a content of the visible light type photoinitiator is preferably 0.1 to 20 parts by mass, further preferably 0.1 to 10 parts by mass, particularly preferably 2 to 4 parts by mass, based on 100 parts by mass of the component (A). Such a range hardly causes yellowing of a cured product. In a case where the visible light type photoinitiator is a mixture of two or more kinds thereof, it is a referred to as a ratio of the total amount of such visible light type photoinitiators. The visible light type photoinitiator as used herein is a photoinitiator which most strongly absorbs light in the visible region and which mainly represents an acyl phosphine oxide-based photopolymerization initiator containing a phosphorus atom. Specific examples thereof may include 2,4,6-trimethylbenzoyl-diphenyl-phosphine oxide and bis(2,4,6-trimethylbenzoyl)-phenylphosphine oxide, but not limited thereto.

The component (D) usable in the present invention is a storage stabilizer having pKa (acid dissociation constant) of 1.0 to 4.0, and does not contain the component (A) and the component (C). More specifically, the component (D) is an organic acid or an inorganic acid having pka of 1.0 to 4.0. Although a reason cannot be clearly found, such an acid is added to the composition according to the present invention to result in enhancements in both storage stability such as viscosity, and surface curability. The pKa is one index quantitatively indicating the strength of an acid (ease of dissociation of a hydrogen ion), and is represented by a negative common logarithm of equilibrium constant (Ka) of dissociation reaction where a proton is released from an acid. A lower pKa can be said to mean a stronger acid. The pKa is known to be measured by neutralization titration, absorptiometry, capillary electrophoresis, or the like, and in particular, neutralization titration has the highest accuracy. The pKa of the storage stabilizer as the component (D) is preferably 1.0 or more and less than 2.3, more preferably 1.50 to 2.20 from the viewpoint of a further enhancement in storage stability.

The component (D) is particularly preferably a phosphorus compound as a storage stabilizer. Herein, phosphonic acid and phosphoric acid are collectively called phosphorus compound. A phosphonic acid compound is more preferred. The phosphonic acid compound is a compound represented by R¹—P(═O)(OR²)₂. Here, R¹ is a hydrogen atom, or an organic group which is not bound to a phosphorus atom via an oxygen atom, and each R² is independently a hydrogen atom or an organic group. Each R² may be the same or different. R¹ may be the same as or different from R². In other words, in a preferred embodiment of the present invention, the component (D) is a phosphorus compound, and the phosphorus compound is a phosphonic acid compound of formula: R¹—P(═O)(OR²)₂, wherein R¹ is a hydrogen atom, or an organic group which is not bound to a phosphorus atom via an oxygen atom, and each R² is independently a hydrogen atom or an organic group. In the formula, examples of the organic group may include an aromatic group such as a phenyl group or a naphthyl group, and a hydrocarbon group, but not limited thereto. Examples of the hydrocarbon group may include an alkyl group, an alkenyl group, and an alkynyl group. Examples of the alkyl group may include a methyl group, an ethyl group, a n-propyl group, an isopropyl group, a n-butyl group, an isobutyl group, a sec-butyl group, a tert-butyl group, a n-pentyl group, an isopentyl group, a tert-pentyl group, a neopentyl group, a 1,2-dimethylpropyl group, a n-hexyl group, an isohexyl group, a 1,3-dimethylbutyl group, a 1-isopropylpropyl group, a 1,2-dimethylbutyl group, a n-heptyl group, a 1,4-dimethylpentyl group, a 3-ethylpentyl group, a 2-methyl-1-isopropylpropyl group, a 1-ethyl-3-methylbutyl group, a n-octyl group, a 2-ethylhexyl group, a 3-methyl-1-isopropylbutyl group, a 2-methyl-1-isopropyl group, a 1-tert-butyl-2-methylpropyl group, a n-nonyl group, a 3,5,5-trimethylhexyl group, a n-decyl group, an isodecyl group, a n-undecyl group, a 1-methyldecyl group, a n-dodecyl group, a n-tridecyl group, a n-tetradecyl group, a n-pentadecyl group, a n-hexadecyl group, a n-heptadecyl group, and a n-octadecyl group. Examples of the alkenyl group may include a vinyl group, an allyl group, a 1-propenyl group, a 2-butenyl group, a 1,3-butadienyl group, a 2-pentenyl group, and an isopropenyl group. Examples of the alkynyl group may include an ethynyl group and a propargyl group. The hydrocarbon group may have any substituent such as a halogen atom, an amino group, a cyano group, a nitro group, or a hydroxy group. The component (D) is most preferably phenylphosphonic acid. The component (D) may be used singly or in combinations of a plurality thereof, and the composition preferably comprises no storage stabilizer other than the component (D). In general, phosphoric acid is represented by P(═O)(OH)₃, and is different from phosphonic acid, H—P(═O)(OH)₂.

Specific examples of the component (D) may include phosphoric acid, oxalic acid, and a phosphonic acid compound, and in particular, examples of the phosphonic acid compound may include 2-ethylhexyl (2-ethylhexyl)phosphonate, phenylphosphonic acid, vinylphosphonic acid, and methylphosphonic acid, but not limited thereto. The component (D) is particularly preferably the phosphonic acid compound. The component (D) may be used singly or in combinations of a plurality thereof. For no reduction in storage stability with respect to viscosity or the like, it is preferable not to use any organic acid or inorganic acid having pka of more than 4.0 in combination, other than the component (D). In other words, in a preferred embodiment of the present invention, the composition does not comprise any organic acid or inorganic acid having pKa of more than 4.0.

In other words, in a preferred embodiment of the present invention, the component (D) is at least one selected from the group consisting of phosphoric acid, oxalic acid, 2-ethylhexyl (2-ethylhexyl)phosphonate, phenylphosphonic acid, vinylphosphonic acid and methylphosphonic acid. In a more preferred embodiment of the present invention, the component (D) is at least one selected from the group consisting of 2-ethylhexyl (2-ethylhexyl)phosphonate, phenylphosphonic acid, vinylphosphonic acid and methylphosphonic acid. In a further preferred embodiment of the present invention, the component (D) is at least one selected from the group consisting of phenylphosphonic acid, vinylphosphonic acid and methylphosphonic acid. In a particularly preferred embodiment of the present invention, the component (D) is phenylphosphonic acid.

The component (D) is preferably added in an amount of 0.01 to 5.0 parts by mass, more preferably added in an amount of 0.01 to 2.0 parts by mass, most preferably added in an amount of 0.05 to 1.5 parts by mass, based on 100 parts by mass of the component (A). When the amount of the component (D) is 0.01 parts by mass or more, change in viscosity can be suppressed, and when the amount is 5.0 parts by mass or less, surface curability can be maintained. The component (D) is preferably added in an amount of less than 1.5 parts by mass, more preferably 0.05 to 1.0 parts by mass based on 100 parts by mass of the component (A), particularly from the viewpoint of a further enhancement in storage stability (further suppression of change in viscosity).

The component (D) is preferably contained at a proportion of 0.01 to 10.0% by mass, more preferably 0.1 to 5.0% by mass, particularly preferably 0.04 to 1.5% by mass, relative to the entire composition. The component (D) is preferably contained at a proportion of less than 1.00% by mass, more preferably at a proportion of 0.04 to 0.82% by mass, relative to the entire composition, particularly from the viewpoint of a further enhancement in storage stability (further suppression of change in viscosity).

In the present invention, a proper amount of additive(s) such as a coupling agent, an inorganic filling agent or an organic filling agent, a colorant such as a pigment or a dye, an antioxidant, a polymerization inhibitor, a defoaming agent, a leveling agent, a rheology control agent, and a slip agent may be compounded as long as features of the present invention are not impaired. Such an additive is added to obtain a composition or a cured product thereof excellent in resin strength, adhesion strength, workability, storage stability, and the like.

In the present invention, a coupling agent can be added as long as features of the present invention are not impaired. Examples of the coupling agent may include a silane-based coupling agent having an epoxy group, a vinyl group, an acryloyl group or a methacryloyl group, and also a hydrolyzable silane group in combination, a polyorganosiloxane having a phenyl group and a hydrolyzable silyl group, and/or a polyorganosiloxane having an epoxy group and a hydrolyzable silyl group, but not limited thereto. Specific examples of the silane-based coupling agent may include allyltrimethoxysilane, vinyltrimethoxysilane, 3-glycidoxypropyltrimethoxysilane, 3-methacryloxypropyltrimethoxysilane, 3-acryloxypropyltrimethoxysilane, and 3-chloropropyltrimethoxysilane, but not limited thereto.

In the present invention, a filling agent such as an inorganic filling agent or an organic filling agent can be added, as long as features of the present invention are not impaired. Such a filling agent can be added to adjust not only viscous properties/thixotropy, but also curability and toughness. Examples of the inorganic filling agent may include alumina, silica and amorphous silica, but not limited thereto. Examples of the organic filling agent may include a styrene filler, a rubber filler, and a core-shell acrylic filler, but not limited thereto. Specific products of silica may include FUSELEX E-1 manufactured by Tatsumori Ltd. and AO-802 manufactured by Admafine, and specific products of amorphous silica may include Aerosil series, 200 (not treated), R972 (treated with dimethyldichlorosilane), R976 (treated with dimethyldichlorosilane), RY200 (treated with dimethylsilicone), RX200 (treated with hexamethyldisilazane), and R800 (treated with octylsilane) manufactured by Nippon Aerosil Co., Ltd., but not limited thereto.

In the present invention, a slip agent can be added as long as features of the present invention are not impaired. If a slip agent is contained, a smooth surface is obtained by leveling action to result in an enhancement in scratch resistance. The slip agent is not particularly limited, can be, for example, a silicone-based surfactant such as polyester-modified silicone or polyether-modified silicone, and polyether-modified polydimethylsiloxane or polyester-modified polydimethylsiloxane is preferably used.

In a case where the composition contains the slip agent, a content of the slip agent is preferably 0.01 to 5.0 parts by mass, more preferably 0.1 to 1.0 part by mass, based on 100 parts by mass of the component (A).

It is preferable before procedures with the composition according to the present invention to perform sanding of a surface of a human nail by a file or the like and then remove any dust, oil content, water content, and the like by a nail dedicated solvent mainly containing ethanol. In the case of coating with the composition according to the present invention, a coating film having a thickness of 100 to 300 μm before curing can be formed by a pencil or a brush. A primer may also be used in advance in the coating. A method for curing the composition according to the present invention is preferably curing of the composition by irradiation with an active energy ray. An irradiation apparatus used in such curing can be a commercially available UV lamp or LED lamp. An irradiation time is 15 seconds to 120 seconds, and is preferably 20 to 70 seconds in consideration of influence on a finger. A cumulative amount of light is preferably 1 to 30 KJ/m².

A compound having a (meth)acryloyl group is inhibited from being polymerized by the action of oxygen inhibition in a region where the compound is in contact with oxygen. The photocurable composition according to the present invention is unlikely to be affected by oxygen inhibition and has rapid curability by light irradiation, and thus is suitable for a nail or an artificial nail, particularly a topcoat for a nail or an artificial nail. In other words, in one embodiment of the present invention, the photocurable composition according to the present invention is used for a nail or an artificial nail. In one embodiment of the present invention, the photocurable composition according to the present invention is used for a nail or an artificial nail, and is for a topcoat (the photocurable composition according to the present invention is a photocurable composition for a topcoat for use in a nail or an artificial nail).

### EXAMPLES

Next, the present invention is further specifically described with reference to Examples, but the present invention is not limited only to these Examples.

### Examples 1 to 11, Comparative Examples 1 to 14, and Reference Examples 1 to 3

The following components were provided for preparation of a photocurable composition (hereinafter, the photocurable composition being also simply referred to as “composition”.)

### Component (A): Compound Having a (Meth)Acryloyl Group

Polyether-based urethane acrylate oligomer having a weight average molecular weight of 5000 and the number of functional groups of 3 (KY-11 manufactured by Negami Chemical Industrial Co., Ltd.) (in the form of a liquid under an atmosphere at 25° C.)
Urethane acrylate oligomer having a weight average molecular weight of 1500 and the number of functional groups of 6 (UN-3320HA manufactured by Negami Chemical Industrial Co., Ltd.) (in the form of a liquid under an atmosphere at 25° C.)
Urethane acrylate oligomer having a weight average molecular weight of 4900 and the number of functional groups of 10 (UN-904 manufactured by Negami Chemical Industrial Co., Ltd.) (in the form of a liquid under an atmosphere at 25° C.)
Polycarbonate-based urethane acrylate oligomer having a weight average molecular weight of 4500 and the number of functional groups of 2 (UF-8001G, Kyoeisha Chemical Co., Ltd.) (in the form of a liquid under an atmosphere at 25° C.)
Dimethylol tricyclodecane diacrylate (Light Acrylate DCP-A manufactured by Kyoeisha Chemical Co., Ltd.) (in the form of a liquid under an atmosphere at 25° C.)
2-Hydroxypropyl methacrylate (HPMA manufactured by Nippon Shokubai Co., Ltd.) (in the form of a liquid under an atmosphere at 25° C.)

### Component (B): Polythiol Compound

Trimethylolpropane tris(3-mercaptopropionate) (TMMP-20P manufactured by SC Organic Chemical Co., Ltd.)

### Component (C): Photoinitiator

1-Hydroxycyclohexyl phenyl ketone (non-visible light type photoinitiator) (IRGACURE184 manufactured by BASF SE)
2,4,6-Trimethylbenzoyl-diphenyl-phosphine oxide (visible light type photoinitiator) (LUCIRIN TPO manufactured by BASF SE)

### Component (D): Phosphorus Compound as Storage Stabilizer

Phenylphosphonic acid (reagent)
Vinylphosphonic acid (reagent)
Methylphosphonic acid (reagent)
Phosphoric acid (reagent)
Oxalic acid (reagent)
   Component (D′): Storage Stabilizer Other than Component (D)
Acetic acid (reagent)
Acrylic acid (reagent)
Benzoic acid (reagent)
Dibutylhydroxytoluene (BHT) (reagent) (antioxidant)
4-Methoxyphenol (MEHQ) (reagent) (polymerization inhibitor)
Hindered phenol-based antioxidant having a molecular weight of 1178 (ADK STAB AO-60 manufactured by ADEKA Corporation)
Dilauryl thiodipropionate (DLTP “Yoshitomi” manufactured by API Corporation) (antioxidant)

### Other

Polyether-modified silicone (LS-480 manufactured by Kusumoto Chemicals Ltd.)

Compositions of Examples 1 to 5 and Comparative Examples 1 to 3 were prepared. The component (A), the component (B), the component (D) (or component (D′)) and other were weighed in a stirring pot and then stirred for 30 minutes. Thereafter, stirring was performed for 30 minutes with defoaming in vacuum. Finally, the component (C) was weighed and added to the stirring pot and the resultant was stirred for 30 minutes. The detailed amounts to be prepared are as shown in Table 1, and all numerical values are expressed by “part(s) by mass”.

TABLE 1

| | | | | | | | | Comparative | Comparative | Comparative |
|---|---|---|---|---|---|---|---|---|---|---|
| Component | Raw material | pKa | Example 1 | Example 2 | Example 3 | Example 4 | Example 5 | Example 1 | Example 2 | Example 3 |
| Component | KY-11 | — | 60 | 60 | 60 | 60 | 60 | 60 | 60 | 60 |
| (A) | DCP-A | — | 10 | 10 | 10 | 10 | 10 | 10 | 10 | 10 |
| | HPMA | — | 30 | 30 | 30 | 30 | 30 | 30 | 30 | 30 |
| Component | TMMP-20P | — | 16.5 | 16.5 | 16.5 | 16.5 | 16.5 | 16.5 | 16.5 | 16.5 |
| (B) | | | | | | | | | | |
| Component | 184 | — | 2.5 | 2.5 | 2.5 | 2.5 | 2.5 | 2.5 | 2.5 | 2.5 |
| (C) | TPO | — | 2.5 | 2.5 | 2.5 | 2.5 | 2.5 | 2.5 | 2.5 | 2.5 |
| Component | Phenylphosphonic | 1.83 | 0.1 | | | | | | | |
| (D) | acid | | | | | | | | | |
| | Vinylphosphonic | 2.11 | | 0.1 | | | | | | |
| | acid | | | | | | | | | |
| | Methylphosphonic | 2.38 | | | 0.1 | | | | | |
| | acid | | | | | | | | | |
| | Phosphoric acid | 1.83 | | | | 0.1 | | | | |
| | Oxalic acid | 1.04 | | | | | 0.1 | | | |
| Component | Acetic acid | 4.76 | | | | | | 0.1 | | |
| (D′) | Acrylic acid | 4.25 | | | | | | | 0.1 | |
| | Benzoic acid | 4.20 | | | | | | | | 0.1 |
| Other | LS-480 | — | 0.12 | 0.12 | 0.12 | 0.12 | 0.12 | 0.12 | 0.12 | 0.12 |

| | | | | | | | | |
|---|---|---|---|---|---|---|---|---|
| Total | 121.7 | 121.7 | 121.7 | 121.7 | 121.7 | 121.7 | 121.7 | 121.7 |
| Proportion (%) of component | 0.08 | 0.08 | 0.08 | 0.08 | 0.08 | 0.08 | 0.08 | 0.08 |
| (D) (component (D′)) relative | | | | | | | | |
| to entire composition | | | | | | | | |

For each of the compositions of Examples 1 to 5 and Comparative Examples 1 to 3, viscosity was measured, surface curability was confirmed and a surface state of cured product was confirmed according to the following methods. The results are summarized in Table 2.

### [Measurement of Viscosity]

Collected was 0.5 ml of each of the compositions, and ejected in a measurement cup. The viscosity was measured with an EHD-type viscometer (manufactured by Tokisangyo) under the following conditions. The result was defined as “viscosity (Pa·s)”. This was defined as initial viscosity. Thereafter, a plastic container filled with each of the compositions was left to still stand in a hot air drying furnace under an atmosphere at 60° C., and viscosity after 2 days was measured. In a case where gel was formed when the container was opened, “Gel formed” was described and no viscosity measurement was performed. In a case where the component(s) was not dissolved, “Not dissolved” was described and no viscosity measurement was performed. The viscosity (initial) is preferably 10 Pa·s or less, more preferably 5 Pa·s or less, from the viewpoint of flowability or the like in consideration of handling during procedures. The rate of change is preferably 20% or less, more preferably 5.0% or less, particularly preferably 3.0% or less.

### Measurement Conditions

Cone rotor: 3°×R14
Rotation rate: 100 rpm
Measurement time: 3 minutes
Measurement temperature: 25° C. (temperature control by constant-temperature bath).

### [Confirmation of Surface Curability]

An acrylic plate of 2.0 mm thickness×25 mm width×100 mm length was coated with the composition by a brush at a thickness of about 300 μm. The composition was cured by irradiation with an UV lamp for nails (rated voltage: 100 to 110 V, consumed power at 50 to 60 Hz: 36 W, wavelength: 350 to 400 nm) for 30 seconds. A surface of a cured product at the time of curing was contacted with a polytetrafluoroethylene bar and a state thereof was visually confirmed according to the following evaluation criteria and then defined as “surface curability”.

### Evaluation Criteria

Good: no occurrence of tackiness of any component on the surface
Poor: occurrence of tackiness of component(s) on the surface.

### [Confirmation of Surface State of Cured Product]

Before the surface curability was confirmed, a surface state was visually confirmed by reflection to a LED stand and evaluated as “Surface of cured product”, according to the following evaluation criteria.

### Evaluation Criteria

Good: glossy surface
Poor: glossless surface

TABLE 2

| | | | | | | | Comparative | Comparative | Comparative |
|---|---|---|---|---|---|---|---|---|---|
| Test item | | Example 1 | Example 2 | Example 3 | Example 4 | Example 5 | Example 1 | Example 2 | Example 3 |
| Viscosity | Initial | 1.60 | 1.73 | 1.71 | 1.80 | 1.72 | 1.73 | 1.72 | 1.70 |
| | After two days | 1.62 | 1.81 | 1.83 | 1.88 | 1.78 | Gel formed | Gel formed | Gel formed |
| | at 60° C. | | | | | | | | |
| | Rate (%) of | 1.3 | 4.6 | 7.0 | 4.4 | 3.5 | — | — | — |
| | change | | | | | | | | |
| Surface | Initial | Good | Good | Good | Good | Good | Good | Good | Good |
| curability | | | | | | | | | |
| Surface of | Initial | Good | Good | Good | Good | Good | Good | Good | Good |
| cured product | | | | | | | | | |

When the compositions of Examples 1 to 5 each including a storage stabilizer having pka of 1.0 to 4.0 are compared with those of Comparative Examples 1 to 3 each including one having pKa of more than 4.0, it can be noted that there is no difference in influence of surface curability, but there is a large difference in storage stability.

Each composition was prepared in Examples 6 to 11 and Comparative Examples 4 to 14. The component (A), the component (B), the component (D) (or component (D′)) and other were weighed in a stirring pot and then stirred for 30 minutes. Thereafter, stirring was performed for 30 minutes with defoaming in vacuum. Finally, the component (C) was weighed and added to the stirring pot and the resultant was stirred for 30 minutes. The detailed amounts to be prepared are as shown in Table 3, and all numerical values are expressed by “part(s) by mass”.

TABLE 3

| | | | | | | | | | Comparative | Comparative |
|---|---|---|---|---|---|---|---|---|---|---|
| Component | Raw material | Example 6 | Example 1 | Example 7 | Example 8 | Example 9 | Example 10 | Example 11 | Example 4 | Example 5 |
| Component | KY-11 | 60 | 60 | 60 | 60 | 60 | 60 | 60 | 60 | 60 |
| (A) | DCP-A | 10 | 10 | 10 | 10 | 10 | 10 | 10 | 10 | 10 |
| | HPMA | 30 | 30 | 30 | 30 | 30 | 30 | 30 | 30 | 30 |
| Component | TMMP-20P | 16.5 | 16.5 | 16.5 | 16.5 | 16.5 | 16.5 | 16.5 | 16.5 | 16.5 |
| (B) | | | | | | | | | | |
| Component | 184 | 2.5 | 2.5 | 2.5 | 2.5 | 2.5 | 2.5 | 2.5 | 2.5 | 2.5 |
| (C) | TPO | 2.5 | 2.5 | 2.5 | 2.5 | 2.5 | 2.5 | 2.5 | 2.5 | 2.5 |
| Component | Phenylphosphonic | 0.05 | 0.1 | 0.2 | 0.3 | 0.5 | 1.0 | 1.5 | | |
| (D) | acid | | | | | | | | | |
| Component | BHT | | | | | | | | | 0.1 |
| (D′) | MEHQ | | | | | | | | | |
| | AO-60 | | | | | | | | | |
| | DLTP | | | | | | | | | |
| | LS-480 | 0.12 | 0.12 | 0.12 | 0.12 | 0.12 | 0.12 | 0.12 | 0.12 | 0.12 |

| | | | | | | | | | |
|---|---|---|---|---|---|---|---|---|---|
| Total | 121.7 | 121.7 | 121.8 | 121.9 | 122.1 | 122.6 | 123.1 | 121.6 | 121.7 |
| Proportion (%) of component | 0.04 | 0.08 | 0.16 | 0.25 | 0.41 | 0.82 | 1.22 | 0.00 | 0.08 |
| (D) (component (D′)) relative | | | | | | | | | |
| to entire composition | | | | | | | | | |

| | | Compar- | Compar- | Compar- | Compar- | Compar- | Compar- | Compar- | Compar- | Compar- |
|---|---|---|---|---|---|---|---|---|---|---|
| | | ative | ative | ative | ative | ative | ative | ative | ative | ative |
| | | Exam- | Exam- | Exam- | Exam- | Exam- | Exam- | Exam- | Exam- | Exam- |
| Component | Raw material | ple 6 | ple 7 | ple 8 | ple 9 | ple 10 | ple 11 | ple 12 | ple 13 | ple 14 |
| Component | KY-11 | 60 | 60 | 60 | 60 | 60 | 60 | 60 | 60 | 60 |
| (A) | DCP-A | 10 | 10 | 10 | 10 | 10 | 10 | 10 | 10 | 10 |
| | HPMA | 30 | 30 | 30 | 30 | 30 | 30 | 30 | 30 | 30 |
| Component | TMMP-20P | 16.5 | 16.5 | 16.5 | 16.5 | 16.5 | 16.5 | 16.5 | 16.5 | 16.5 |
| (B) | | | | | | | | | | |
| Component | 184 | 2.5 | 2.5 | 2.5 | 2.5 | 2.5 | 2.5 | 2.5 | 2.5 | 2.5 |
| (C) | TPO | 2.5 | 2.5 | 2.5 | 2.5 | 2.5 | 2.5 | 2.5 | 2.5 | 2.5 |
| Component | Phenylphosphonic | | | | | | | | | |
| (D) | acid | | | | | | | | | |
| Component | BHT | 0.5 | | | | | | | | |
| (D′) | MEHQ | | 0.1 | 0.5 | | | | | | |
| | AO-60 | | | | 0.5 | | 0.5 | 0.1 | 1.0 | |
| | DLTP | | | | | 0.5 | 0.5 | 0.1 | | 1.0 |
| | LS-480 | 0.12 | 0.12 | 0.12 | 0.12 | 0.12 | 0.12 | 0.12 | 0.12 | 0.12 |

| | | | | | | | | | |
|---|---|---|---|---|---|---|---|---|---|
| Total | 122.1 | 121.7 | 122.1 | 122.1 | 122.1 | 122.6 | 121.8 | 122.6 | 122.6 |
| Proportion (%) of component | 0.41 | 0.08 | 0.41 | 0.41 | 0.41 | 0.82 | 0.16 | 0.82 | 0.82 |
| (D) (component (D′)) relative | | | | | | | | | |
| to entire composition | | | | | | | | | |

For each of the compositions of Examples 1, 6 to 11 and Comparative Examples 4 to 14, viscosity was measured and surface curability was confirmed according to the following methods. The results are summarized in Table 4.

### [Measurement of Viscosity]

Collected was 0.5 ml of each of the compositions, and ejected in a measurement cup. The viscosity was measured with an EHD-type viscometer (manufactured by Tokisangyo) under the following conditions. The result was defined as “viscosity (Pa·s)”. This was defined as initial viscosity. Thereafter, a plastic container filled with each of the compositions was left to still stand in a hot air drying furnace under an atmosphere at 40° C., and viscosity after 21 days was measured. In a case where gel was formed when the container was opened, “Gel formed” was described and no viscosity measurement was performed. In a case where the component(s) was not dissolved, “Not dissolved” was described and no viscosity measurement was performed. The viscosity (initial) is preferably 10 Pa·s or less, more preferably 5 Pa·s or less from the viewpoint of flowability or the like in consideration of handling during procedures. The rate of change is preferably 20% or less, more preferably less than 10.0%.

### Measurement Conditions

Cone rotor: 3°×R14
Rotation rate: 100 rpm
Measurement time: 3 minutes
Measurement temperature: 25° C. (temperature control by constant-temperature bath).

### [Confirmation of Surface Curability]

An acrylic plate of 2.0 mm thickness×25 mm width×100 mm length was coated with the composition by a brush at a thickness of about 300 μm. The composition was cured by irradiation with an UV lamp for nails (rated voltage: 100 to 110 V, consumed power at 50 to 60 Hz: 36 W, wavelength: 350 to 400 nm) for 30 seconds. A surface of a cured product at the time of curing was contacted with a polytetrafluoroethylene bar and a state thereof was visually confirmed according to the following evaluation criteria and then defined as “surface curability”.

### Evaluation Criteria

Good: no occurrence of tackiness of any component on the surface
Poor: occurrence of tackiness of component(s) on the surface

TABLE 4

| | Raw | | | | | | | | Comparative | Comparative |
|---|---|---|---|---|---|---|---|---|---|---|
| Component | material | Example 6 | Example 1 | Example 7 | Example 8 | Example 9 | Example 10 | Example 11 | Example 4 | Example 5 |
| Viscosity | Initial | 1.62 | 1.60 | 1.60 | 1.66 | 1.76 | 1.76 | 1.76 | 1.61 | 1.61 |
| | After 21 days | 1.70 | 1.70 | 1.73 | 1.76 | 1.81 | 1.87 | 1.95 | Gel formed | 1.65 |
| | at 40° C. | | | | | | | | | |
| | Rate (%) of | 4.9 | 6.2 | 8.1 | 6.2 | 2.7 | 6.3 | 10.8 | — | 2.5 |
| | change | | | | | | | | | |
| Surface | Initial | Good | Good | Good | Good | Good | Good | Good | Good | Poor |
| curability | | | | | | | | | | |

| | | Compar- | Compar- | Compar- | Compar- | Compar- | Compar- | Compar- | Compar- | Compar- |
|---|---|---|---|---|---|---|---|---|---|---|
| | | ative | ative | ative | ative | ative | ative | ative | ative | ative |
| | Raw | Exam- | Exam- | Exam- | Exam- | Exam- | Exam- | Exam- | Exam- | Exam- |
| Component | material | ple 6 | ple 7 | ple 8 | ple 9 | ple 10 | ple 11 | ple 12 | ple 13 | ple 14 |
| Viscosity | Initial | 1.62 | 1.61 | 1.63 | 1.68 | 1.65 | 1.61 | 1.61 | Not | 1.61 |
| | | | | | | | | | dissolved | |
| | After 21 days | 1.65 | 1.71 | 1.70 | Gel | Gel | 2.03 | Gel | — | Gel |
| | at 40° C. | | | | formed | formed | | formed | | formed |
| | Rate (%) of | 1.9 | 6.2 | 4.3 | — | — | 26.1 | — | — | — |
| | change | | | | | | | | | |
| Surface | Initial | Poor | Poor | Poor | Poor | Poor | Poor | Good | Poor | Good |
| curability | | | | | | | | | | |

The compositions of Examples 1 and 6 to 11, in which phenylphosphonic acid was added as the component (D), were found to exhibit a low viscosity change rate after 21 days at 40° C. and “Good” surface curability. On the other hand, the compositions of Comparative Examples 4 to 14, in which a component used as a polymerization inhibitor or an antioxidant was used, did not allow for suppression of polymerization under an atmosphere at 40° C. and caused gel formation, or caused so strong suppression of polymerization that the surface curability was “Poor”. It has been thus noted that the component (D) in the present invention allows both storage stability and surface curability to be specifically achieved.

Each composition where the oligomer was changed was prepared in Reference Examples 1 to 3. The component (A), the component (B), the component (D) and other were weighed in a stirring pot and then stirred for 30 minutes. Thereafter, stirring was performed for 30 minutes with defoaming in vacuum. Finally, the component (C) was weighed and added to the stirring pot and the resultant was stirred for 30 minutes. The detailed amounts to be prepared are as shown in Table 5, and all numerical values are expressed by “part(s) by mass”. The results of the surface curability confirmed above are also described.

TABLE 5

| | | Example | Reference | Reference | Reference |
|---|---|---|---|---|---|
| Component | Raw material | 6 | Example 1 | Example 2 | Example 3 |
| Component (A) | KY-11 | 60 | | | |
| | UN-3320HA | | 60 | | |
| | UN-904 | | | 60 | |
| | UF-8001G | | | | 60 |
| | DCP-A | 10 | 10 | 10 | 10 |
| | HPMA | 30 | 30 | 30 | 30 |
| Component (B) | TMMP-20P | 16.5 | 16.5 | 16.5 | 16.5 |
| Component (C) | 184 | 2.5 | 2.5 | 2.5 | 2.5 |
| | TPO | 2.5 | 2.5 | 2.5 | 2.5 |
| Component (D) | Phenylphosphonic | 0.05 | 0.05 | 0.05 | 0.05 |
| | acid | | | | |
| Other | LS-480 | 0.12 | 0.12 | 0.12 | 0.12 |
| Total | | 121.7 | 121.7 | 121.7 | 121.7 |
| Proportion (%) of component (D) | | 0.04 | 0.04 | 0.04 | 0.04 |
| (component (D′)) relative to | | | | | |
| entire composition | | | | | |
| Surface curability | Initial | Good | Good | Good | Poor |

When the composition of Example 6 is compared with the compositions of Reference Examples 1 to 3, it can be noted that a urethane acrylate oligomer having the number of functional groups of 3 or more in one molecule is preferred in terms of surface curability.

### INDUSTRIAL APPLICABILITY

The present invention is to provide a photocurable composition which, although comprises a polythiol compound, has both surface curability and storage stability (suppression of change in viscosity). In particular, the change in viscosity in procedures in the nail field has an influence on coatability, and the composition according to the present invention can be stably applied in such procedures and can be applied particularly as a topcoat.

The present application is based on Japanese patent application No. 2021-184527 filed on Nov. 12, 2021, the disclosed content of which is incorporated by reference in its entirety.

1. A photocurable composition comprising components (A) to (D):
Component (A): a compound having a (meth)acryloyl group
Component (B): a polythiol compound
Component (C): a photoinitiator
Component (D): a storage stabilizer having pKa of 1.0 to 4.0, containing no component (A).

2. The photocurable composition according to claim 1, wherein the component (D) is a phosphorus compound.

3. The photocurable composition according to claim 2, wherein the phosphorus compound is a phosphonic acid compound R¹—P(═O)(OR²)₂, wherein R¹ is hydrogen, or an organic group not bound to a phosphorus atom via an oxygen atom, and each R² is independently a hydrogen atom or an organic group.

4. The photocurable composition according to claim 1, wherein the component (D) is at least one selected from the group consisting of phosphoric acid, oxalic acid, 2-ethylhexyl (2-ethylhexyl)phosphonate, phenylphosphonic acid, vinylphosphonic acid and methylphosphonic acid.

5. The photocurable composition according to claim 1, comprising 0.01 to 10.0% by mass of the component (D) relative to the entire composition.

6. The photocurable composition according to claim 1, wherein the composition does not comprise any storage stabilizer other than the component (D).

7. The photocurable composition according to claim 1, comprising 0.1 to 50 parts by mass of the component (B) and 0.1 to 10 parts by mass of the component (C), based on 100 parts by mass of the component (A).

8. The photocurable composition according to claim 1, wherein the component (A) comprises a (meth)acrylate oligomer and a (meth)acrylate monomer.

9. The photocurable composition according to claim 8, wherein the (meth)acrylate monomer consists only of a monofunctional (meth)acrylate and/or a difunctional (meth)acrylate.

10. The photocurable composition according to claim 9, wherein the monofunctional (meth)acrylate is a monofunctional (meth)acrylate having a hydroxyl group.

11. The photocurable composition according to claim 9, wherein the difunctional (meth)acrylate is dimethylol tricyclodecane diacrylate.

12. The photocurable composition according to claim 1, for use in a nail or an artificial nail.

13. The photocurable composition for use in a nail or an artificial nail according to claim 12, wherein the photocurable composition is for a topcoat.
